(19) United States
(12) Patent Application Publication
(10) Pub. No.: US 2011/0237952 A1
(43) Pub. Date: Sep. 29, 2011

(54) TWO-DIMENSIONAL-ARRAY ULTRASONIC PROBE AND ULTRASONIC DIAGNOSTIC APPARATUS

(76) Inventors: Michiko Ooishi, Kawasaki-shi (JP); Satoru Asagiri, Yokohama-shi (JP); Takeshi Miyagi, Fujisawa-shi (JP); Takashi Togasaki, Yokohama-shi (JP)

(21) Appl. No.: 13/051,451

(22) Filed: Mar. 18, 2011

(30) Foreign Application Priority Data
Mar. 24, 2010 (JP) 2010-068683

(51) Int. Cl. A61B 8/00

(52) U.S. Cl. 600/459

(57) ABSTRACT

According to one embodiment, an ultrasonic probe comprises piezoelectric elements arranged in the form of a two-dimensional array, a processing IC configured to process signal information obtained from the piezoelectric elements, and a flexible wiring substrate disposed between the piezoelectric elements and the processing IC, with the piezoelectric elements mounted on a front surface, and the processing IC mounted on a rear surface.

### CROSS-REFERENCE TO RELATED APPLICATIONS

This application is based upon and claims the benefit of priority from Japanese Patent Application No. 2010-068683, filed Mar. 24, 2010; the entire contents of which are incorporated herein by reference.

### FIELD

Embodiments described herein relate generally to a two-dimensional-array ultrasonic probe that outputs ultrasonic waves by using piezoelectric elements arranged in the form of a two-dimensional array and receives reflected ultrasonic waves, and an ultrasonic diagnostic apparatus with such a two-dimensional-array ultrasonic probe incorporated therein.

### BACKGROUND

A two-dimensional-array ultrasonic probe is used in an ultrasonic diagnostic apparatus used for a diagnosis of an echo image. The two-dimensional-array ultrasonic probe is an apparatus with piezoelectric elements arranged in a head in the form of a two-dimensional array, so as to output ultrasonic waves from the piezoelectric elements and receive reflected ultrasonic waves, wherein a detected signal is transmitted to an inspection device body, etc., via a cable, which is then subjected to image processing and is used for a diagnosis, etc.

The aforementioned two-dimensional-array ultrasonic probe involves the following problem. Namely, in recent years, a real time diagnosis by a three-dimensional moving image is realized, and in order to obtain a clear image, a design of increasing the number of channels of the piezoelectric elements mounted on a head has been attempted. With such a design, the number of connection wires for connecting to the inspection device body is increased, resulting in a thick cable of these connection wires. In a case of the thick wire, the head of the two-dimensional-array ultrasonic probe is hardly moved, thus unfavorably disturbing the diagnosis.

Therefore, in order to achieve a real time diagnosis by the three-dimensional moving image, and in order to obtain a clear image, it is desired to provide a two-dimensional-array ultrasonic probe easy to be handled with no necessity of making the cable thick even if the number of channels is increased, and an ultrasonic diagnostic apparatus with such a two-dimensional-array ultrasonic probe incorporated therein.

### BRIEF DESCRIPTION OF THE DRAWINGS

FIG. 1 is a perspective view showing an ultrasonic diagnostic apparatus with an ultrasonic probe incorporated therein according to a first embodiment;

FIG. 2 is a perspective view showing the aforementioned ultrasonic probe;

FIG. 3 is an explanatory view showing a detector incorporated in the aforementioned ultrasonic probe;

FIG. 4 is a cross-sectional view showing an essential part of an interposer substrate incorporated in the aforementioned detector;

FIG. 5 is a cross-sectional view showing an essential part of a flexible wiring substrate incorporated in the aforementioned detector;

FIG. 6 is a plan view showing a switch IC incorporated in the aforementioned detector;

FIG. 7 is a flowchart showing manufacturing steps of the aforementioned ultrasonic probe;

FIG. 8 is an explanatory view showing the aforementioned manufacturing steps;

FIG. 9A is an explanatory view showing the aforementioned manufacturing steps;

FIG. 9B is an explanatory view showing the aforementioned manufacturing steps;

FIG. 9C is an explanatory view showing the aforementioned manufacturing steps;

FIG. 10 is an explanatory view showing the aforementioned manufacturing steps;

FIG. 11A is an explanatory view showing the aforementioned manufacturing steps;

FIG. 11B is an explanatory view showing the aforementioned manufacturing steps;

FIG. 12A is an explanatory view showing the aforementioned manufacturing steps;

FIG. 12B is an explanatory view showing the aforementioned manufacturing steps;

FIG. 12C is a vertical cross-sectional view showing the aforementioned manufacturing steps;

FIG. 12D is a vertical cross-sectional view showing the aforementioned manufacturing steps; and

FIG. 13 is an explanatory view showing the detector incorporated in the ultrasonic probe according to a second embodiment.

### DETAILED DESCRIPTION

In general, according to one embodiment, a two-dimensional-array ultrasonic probe comprises: piezoelectric elements arranged in the form of a two-dimensional array; a processing IC for processing signal information obtained from the piezoelectric elements; and a flexible wiring substrate disposed between the piezoelectric elements and the processing IC, with the piezoelectric elements mounted on a front surface, and the processing IC mounted on a rear surface.

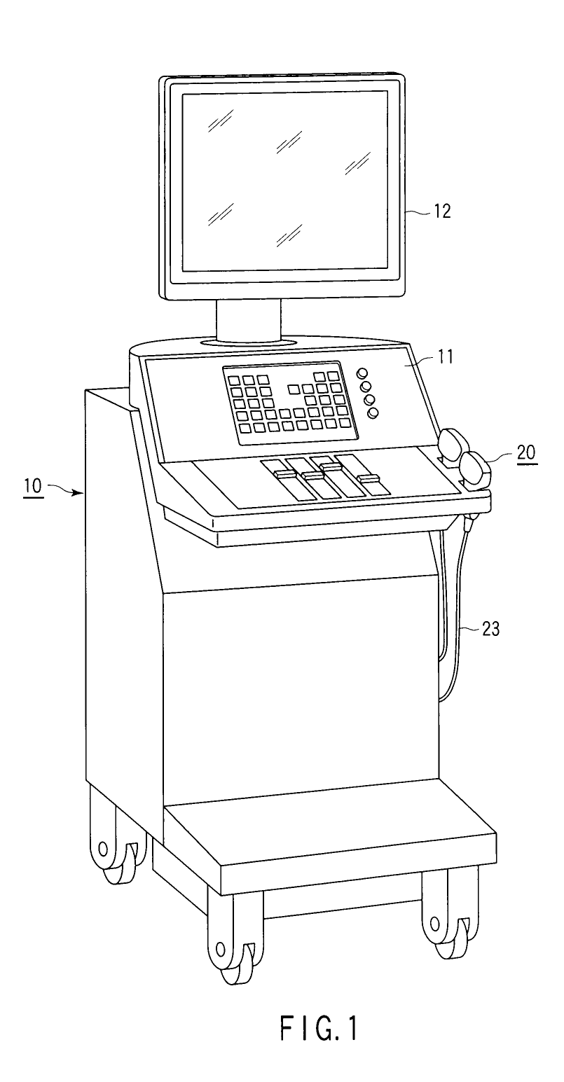

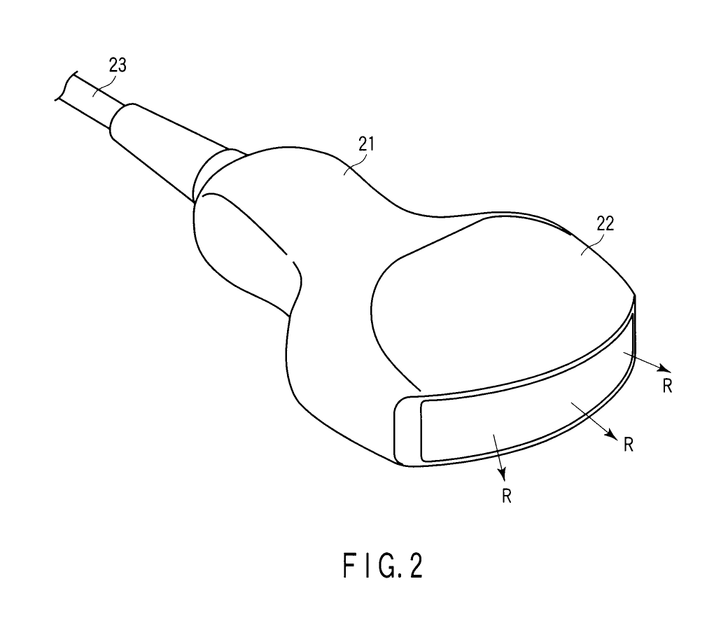

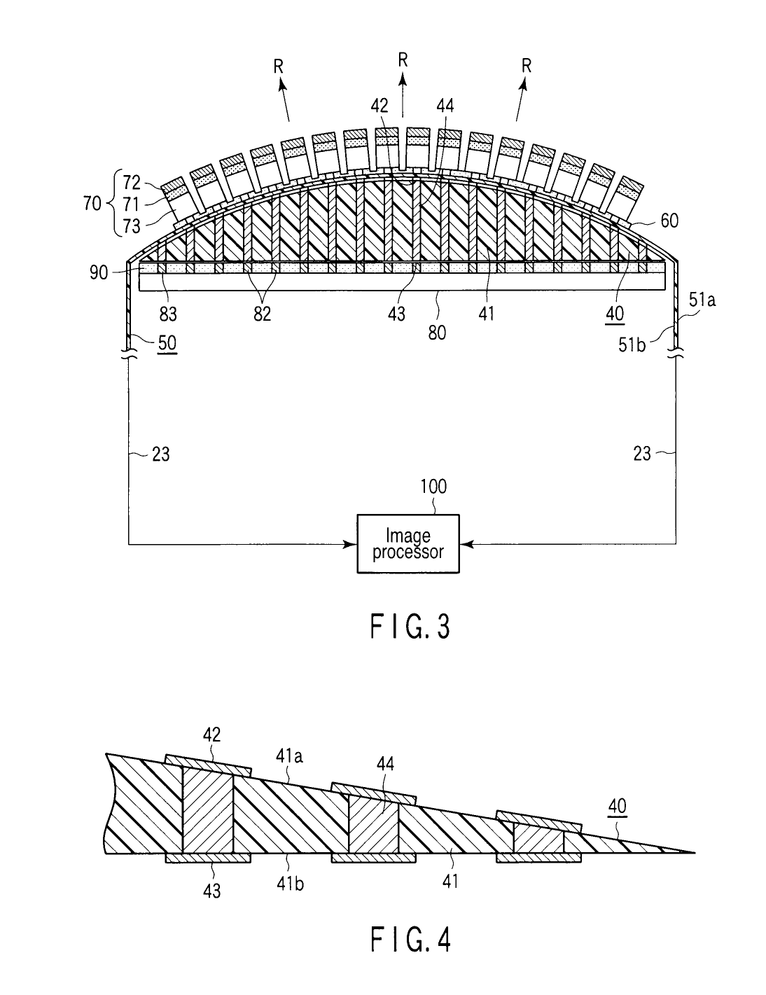

FIG. 1 is a perspective view showing an ultrasonic diagnostic apparatus 10 according to a first embodiment; FIG. 2 is a perspective view showing an ultrasonic probe 20 incorporated in the ultrasonic diagnostic apparatus 10; and FIG. 3 is a cross-sectional view showing a structure of a detector 30 incorporated in the ultrasonic probe 20. Note that R in the figure shows an irradiating direction of ultrasonic waves.

As shown in FIG. 1, the ultrasonic diagnostic apparatus 10 comprises: a diagnostic apparatus body 11; an image monitor 12 attached to the diagnostic apparatus body 11; and an ultrasonic probe (convex two-dimensional-array ultrasonic probe) 20 attached via a cable 13 from the diagnostic apparatus body 11.

An image processor 100 is provided inside of the diagnostic apparatus body 11, for forming an image by processing a signal sent from the ultrasonic probe 20. Further, the image monitor 12 has a function of displaying the image formed by the image processor 100.

As shown in FIG. 2, the ultrasonic probe 20 comprises: a hand portion 21 grasped by an operator; a head 22 in which the detector 30 is accommodated; and a cable 23 for transmitting and receiving signals to/from the diagnostic apparatus body 11. Note that the head 22 has a convex surface in the irradiating direction of the ultrasonic waves (shown by an arrow R in FIG. 2).

As shown in FIG. 3, the detector 30 comprises: an interposer substrate (relay substrate) 40 formed into a convex shape (convex type); a flexible wiring substrate 50 disposed with its rear surface side facing the convex side of the interposer substrate 40; two-dimensional-array piezoelectric elements 70 mounted on the front surface side of the flexible wiring substrate 50 via an adhesive layer 60; and a switch IC (processing IC) 80 mounted on a flat-plate side of the interposer substrate 40 via an adhesive layer 90.

FIG. 4 is a cross-sectional view showing an essential part of the interposer substrate 40. As shown in FIG. 4, the interposer substrate 40 comprises: a base material 41 including a resin material; first electrodes 42 provided on a front surface 41a side of the base material 41; second electrodes 43 provided on a rear surface 41b side; and through electrodes 44 passing through the base material 41 so as to connect the first electrodes 42 and the second electrodes 43. The surface 41a of the base material 41 is formed into a convex shape, and the rear surface 41b is formed into a flat-surface shape.

The first electrodes 42 are connected to second electrodes 53 of the flexible wiring substrate 50, for taking out electrical wires via the second electrodes 53. The second electrodes 43 are provided on an opposite surface to the first electrodes 42, and are electrically connected to the switch IC 80.

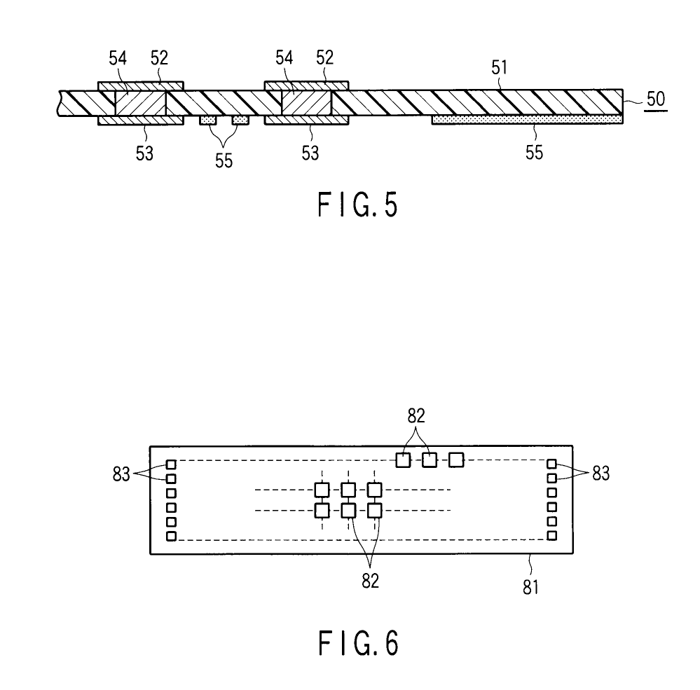

FIG. 5 is a cross-sectional view showing an essential part of the flexible wiring substrate 50. The flexible wiring substrate 50 comprises: a base material 51 including a resin material such as polyimide having flexibility; first electrodes 52 provided on a front surface side of the base material 51; second electrodes 53 provided on a rear surface side; through electrodes 54 passing through the base material 51 so as to connect the first electrodes 52 and the second electrodes 53; and a wiring part 55 such as a copper foil.

The first electrodes 52 are connected to the piezoelectric elements 70, and take out the electrical wires from lower side electrodes (not shown) of the piezoelectric elements 70. The second electrodes 53 are connected to the first electrodes 42 of the interposer substrate 40. The wiring portion 55 is pulled out to outside of a connection area connected to the piezoelectric elements 70 and the interposer substrate 40, and is connected to the image processor 100 via the cable 23.

An arrangement pitch of the first electrodes 52 is 400 μm for example, and an interval between adjacent first electrodes 52 is 80 μm. The base material is preferably formed as a thin base, from a point that bending property is required. Further, a bump 52a with a height of about 40 μm (Cu core, surface treatment: Ni/Au plating) is formed in each of the first electrodes 52.

The adhesive layer 60 has not only a function of preventing the piezoelectric elements 70 from being peeled off in a dicing step of the piezoelectric elements 70 as described later, but also a function of sufficiently securing a depth of dicing so that the piezoelectric elements 70 are cut off by a blade up to a middle thereof in a direction of a thickness (namely, they are not completely cut off).

Two-dimensional-array piezoelectric elements 70 are arranged in the form of a two-dimensional array, and with the appearance of a convex curved surface, wherein a piezoelectric vibrator 71, an acoustic matching layer 72, and a backing material 73 are formed by lamination (see FIG. 8). A dimension of the piezoelectric elements 70 is 60 mm×10 mm for example.

The piezoelectric vibrator 71 includes an upper side electrode and a lower side electrode (each of them is not shown) attached to piezoelectric ceramics, etc., such as lead zirconate titanate (PZT). The piezoelectric vibrator 71 has a function of generating ultrasonic waves based on a driving signal from a pulser, and converting a reflected wave to an electrical signal, the reflected wave being reflected from an inspection target.

The acoustic matching layer 72 can perform matching of acoustic impedance between the inspection target and the piezoelectric vibrator 71 by adjusting physical parameters such as sound speed, thickness, and acoustic impedance.

In order to shorten an ultrasonic wave pulse, the backing material 73 has a function of mechanically supporting the piezoelectric vibrator 71 and putting a brake on the piezoelectric vibrator 71. Also, in order to favorably maintain acoustic properties, a thickness of the backing material 73 is set to a sufficient thickness (specifically, a thickness capable of sufficiently attenuating the ultrasonic wave in a back face direction) with respect to a wavelength of an ultrasonic wave to be used.

As shown in FIG. 6, the switch IC 80 comprises: an IC main body 81; area electrodes 82 for inputting the electrical signal received from the piezoelectric elements 70; and external electrodes 83 for outputting the electrical signal that has undergone signal processing. Although electrical signals received from a plurality of piezoelectric vibrators 71 are input into the switch IC 80 respectively, the electrical signals are output after being converted to signals for generating images. Therefore, the number of output signals can be drastically reduced.

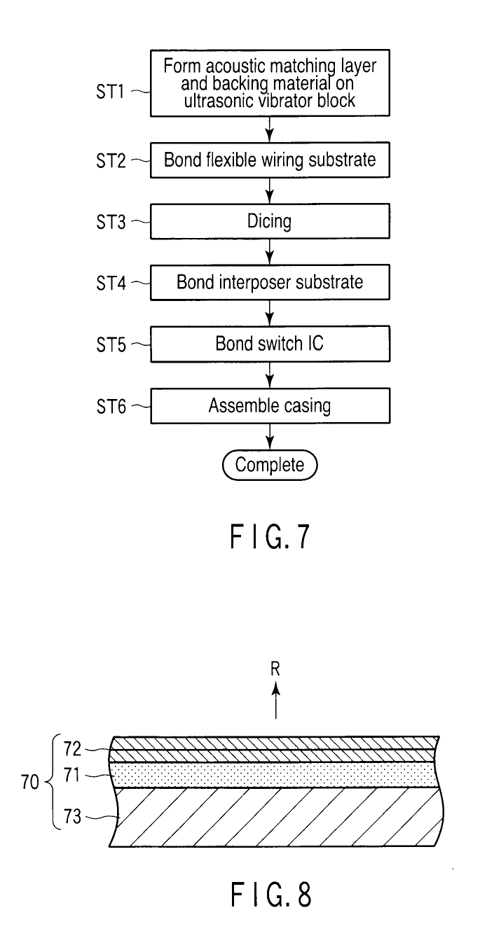

Next, manufacturing steps of such an ultrasonic probe 20 will be described with reference to a flowchart shown in FIG. 7. First, as shown in FIG. 8, the upper side electrode and the lower side electrode are attached for applying a voltage to the piezoelectric vibrator 71, and the acoustic matching layer 72 is formed on the upper side electrode, and the backing material 73 is formed on the lower side electrode (ST1).

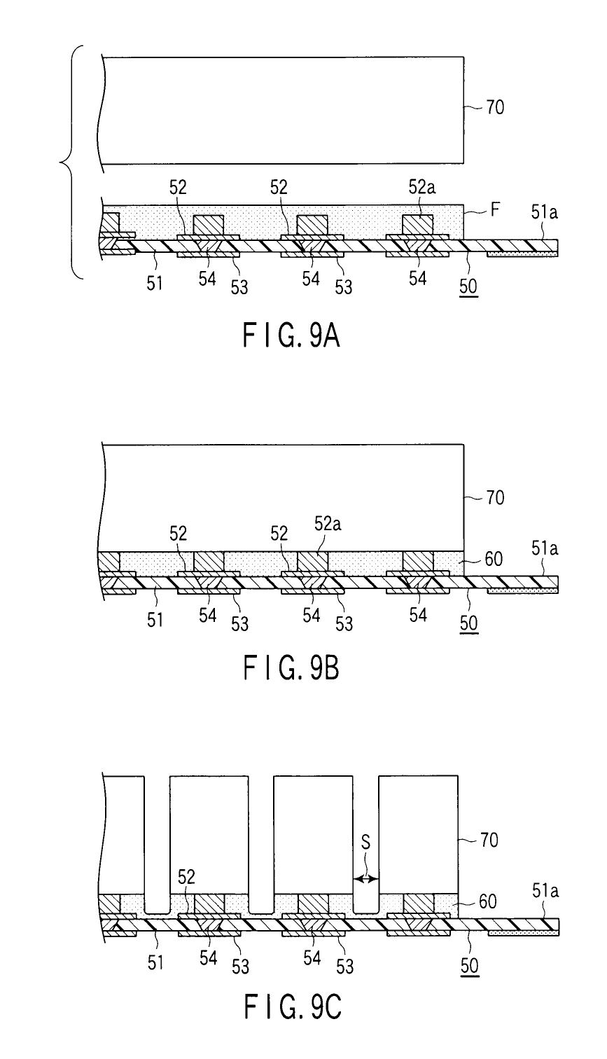

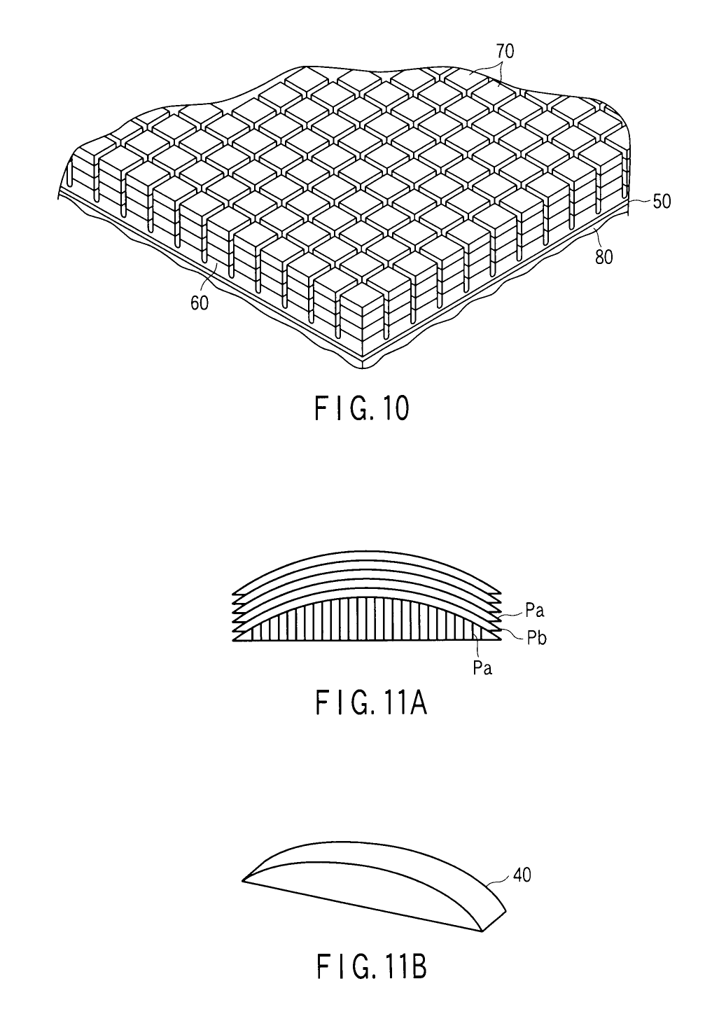

Next, as shown in FIG. 9A, an anisotropic electroconductive film F, being a base material of the adhesive layer 60, is laminated on a front surface 51a side of the flexible wiring substrate 50. Then, as shown in FIG. 9B, the piezoelectric body 70 is aligned at a specified position, and is bonded thereto by using a thermo-compression bonding apparatus (not shown) (ST2). Next, as shown in FIG. 9C, the piezoelectric elements 70 bonded to the flexible wiring substrate 50 are temporarily fixed to a cutting base, and dicing is performed thereto at an interval of 400 μm by using a blade of 50 μm (ST3). At this time, a cutting depth is set so that the adhesive layer 60 is cut up to about 20 μm, so that the piezoelectric elements 70 are surely cut. Thereafter, the temporarily fixed flexible wiring substrate 50 (the piezoelectric body 70 is bonded thereto) is removed from the cutting base. FIG. 10 is a perspective view showing the piezoelectric elements 70 and the adhesive layer 60 after dicing.

Meanwhile, the interposer substrate 40 is formed as shown in FIG. 11A and FIG. 11B. Namely, as shown in FIG. 11A, a printed wiring substrate of 36 layers is prepared, wherein 36 sheets of substrates are laminated on each other with wiring patterns formed thereon so as to correspond to through electrodes. Next, an outer shape is ground, to thereby form the interposer substrate 40 as shown in FIG. 11B. Thereafter, total surface plating (such as Ni/Au) and patterning (exposure, developing, or cutting) may be applied to the first electrodes 42 and the second electrodes 43, as a surface treatment.

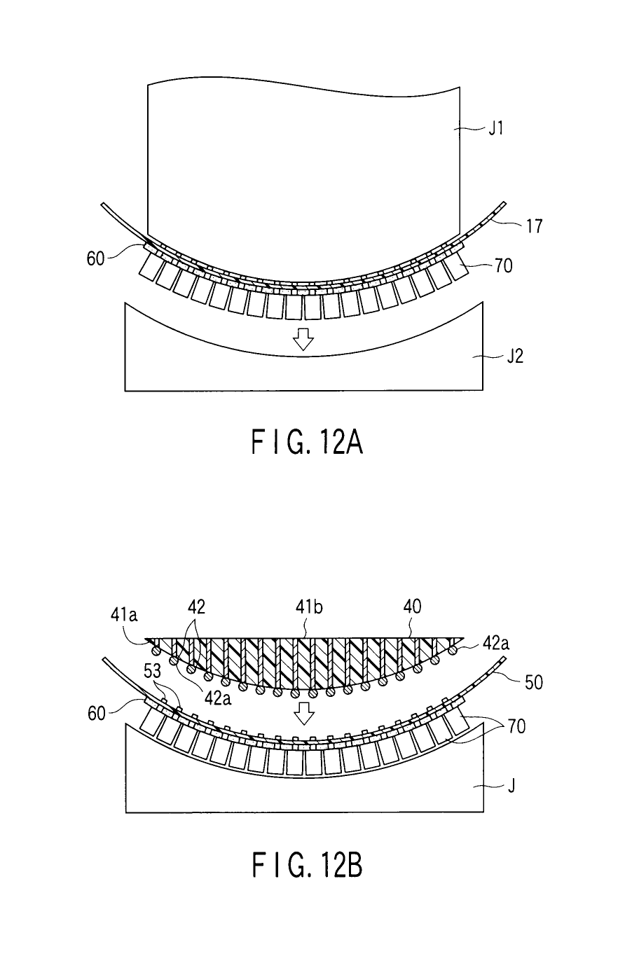

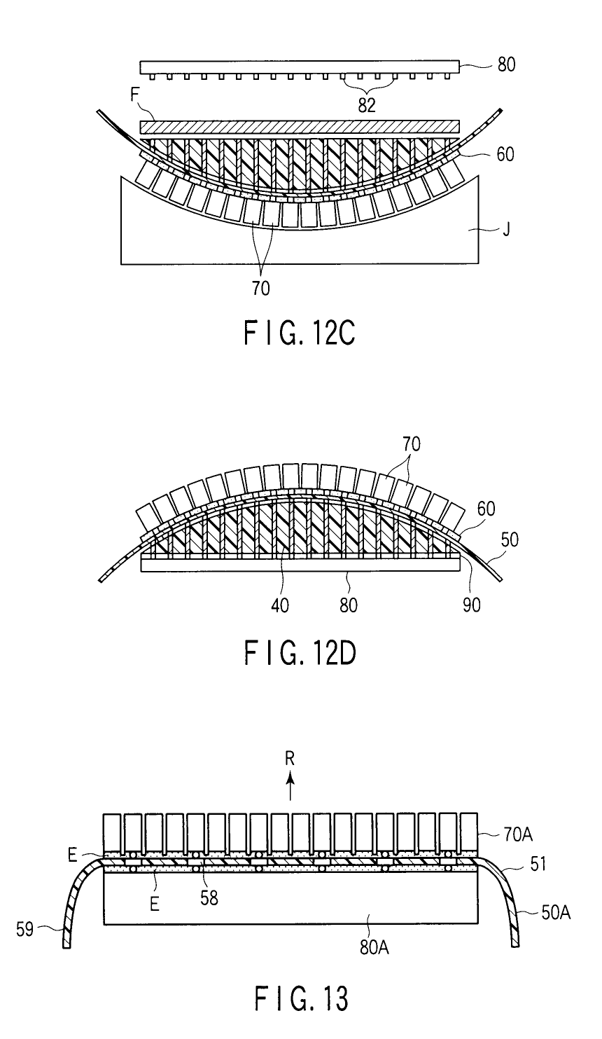

Next, as shown in FIG. 12A, the piezoelectric elements 70 are arranged with the appearance of a convex shape over an irradiation surface of the ultrasonic wave by using a jig J1 having a convex curved surface and a jig J2 having a concave curved surface.

Next, as shown in FIG. 12B, solders 42a are formed in advance on the first electrodes 42 of the interposer substrate 40, so that the second electrodes 53 of the flexible wiring substrate 50 and the first electrodes 42 of the interposer substrate 40 are connected to each other by soldering (ST4).

Next, as shown in FIG. 12C and FIG. 12D, the anisotropic electroconductive film F is laminated on the interposer substrate 40, then, the switch IC 80 with an Au bump previously formed on the electrode is aligned at a position of the interposer substrate 40, to thereby connect the interposer substrate 40 and the switch IC 80 by using the thermal compression bonding apparatus N (ST5).

Thereafter, this is incorporated in a casing (ST6), and the ultrasonic probe 20 is completed.

As described above, in the ultrasonic probe 20 according to this embodiment, by using the interposer substrate 40 with one surface formed into the convex curved surface and having through electrodes, the switch IC 80 for processing huge quantities of signal information obtained from the piezoelectric elements can be connected to the vicinity of the piezoelectric elements 70. Therefore, the real time diagnosis by the three-dimensional moving image is possible, and even when the number of the piezoelectric body is increased to obtain a clear image, the number of signal cable connected to the image processor 100 can be reduced. Accordingly, the thickness of the cable 23 can be made small, thus making it easy to handle the head 22.

FIG. 13 is an explanatory view showing a structure of an ultrasonic probe 20A according to a second embodiment. Note that in FIG. 13, the same signs and numerals are assigned to the same functional parts as those of FIG. 3, and detailed explanation thereof is omitted. The ultrasonic probe 20A comprises a detector 30A.

The detector 30A comprises: two-dimensional-array piezoelectric elements 70A which are arranged with the appearance of a flat-plate shape; a flexible wiring substrate 50A with the piezoelectric elements 70A mounted on a front surface side via an adhesive layer E; and a switch IC 80A connected to a rear surface side of the flexible wiring substrate 50A via the adhesive layer E.

Thus, when the two-dimensional-array piezoelectric elements 70A are arranged with the appearance of a flat-plate shape, the detector 30A can be formed, with an interposer substrate omitted. In the detector 30A with such a structure, signals obtained by the piezoelectric elements 70 can be sent to an image processor 100 via the switch IC 80A, thus making it possible to reduce the number of signal cables, and possible to make the thickness of the cable 23 small.

Note that in an example described above, a gold bump and the anisotropic electroconductive film are used as connection materials. However, for example, an electroconductive adhesive agent or solder, etc., may also be used, and further underfill materials may also be properly used. In addition, grooves provided to the piezoelectric elements may also be filled with epoxy resin, etc. Further, the switch IC is given as an example of the processing IC. However, other processing IC such as control IC may also be used.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasonic probe comprising:
piezoelectric elements arranged in the form of a two-dimensional array;
a processing IC configured to process signal information obtained from the piezoelectric elements; and
a flexible wiring substrate disposed between the piezoelectric elements and the processing IC, with the piezoelectric elements mounted on a front surface, and the processing IC mounted on a rear surface.

2. An ultrasonic probe comprising:
piezoelectric elements arranged in the form of a two-dimensional array and with the appearance of a convex curved-surface shape;
a relay substrate including a substrate main body with a front surface formed into a convex curved-surface shape along the piezoelectric elements, and a rear surface formed into a flat-surface shape, a front surface electrode formed on the front surface, a rear surface electrode formed on the rear surface, and a through electrode passing through from the front surface electrode to the rear surface electrode;
a flexible wiring substrate disposed between the piezoelectric elements and the relay substrate, with the piezoelectric elements mounted on a front surface, and electrodes of the relay substrate connected to a rear surface; and
a processing IC mounted on the rear surface electrode of the relay substrate and configured to process signal information obtained from the piezoelectric elements.

3. An ultrasonic diagnostic apparatus comprising:
an ultrasonic probe including piezoelectric elements arranged in the form of a two-dimensional array, a processing IC configured to process signal information obtained from the piezoelectric elements, and a flexible wiring substrate disposed between the piezoelectric elements and the processing IC, with the piezoelectric elements mounted on a front surface, and the processing IC mounted on a rear surface;
an image processor configured to process a signal sent from the processing IC and form an image; and
an image displayer configured to display the image formed by the image processor.
